(19) United States
(12) Patent Application Publication
(10) Pub. No.: US 2022/0315986 A1
(43) Pub. Date: Oct. 6, 2022

(54) PROCESSES FOR ENRICHING DESIRABLE ELEMENTS AND USES THEREFOR

(71) Applicant: Diversity Arrays Technology Pty Limited, Bruce (AU)

(72) Inventor: Andrzej Kilian, Bruce (AU)

(21) Appl. No.: 17/700,214

(22) Filed: Mar. 21, 2022

(30) Foreign Application Priority Data
Apr. 1, 2021 (AU) 2021900974

(51) Int. Cl. C12Q 1/6806

(52) U.S. Cl. CPC C12Q 1/6806

(57) ABSTRACT

Disclosed herein are processes for enriching low-copy elements in DNA samples. More particularly, the present disclosure relates to the use of a methylation-dependent restriction endonuclease that recognizes methylated DNA and that cleaves upstream and downstream of the methylated DNA in processes for depleting repetitive methylated DNA elements and for enriching low-copy elements. The disclosed processes have particular utility in methods for analyzing features of low-copy elements with increased sensitivity.

### RELATED APPLICATIONS

This application claims priority to Australian Provisional Application No. 2021900974 entitled “Pathogen moieties and uses thereof” filed 1 Apr. 2022, the contents of which are incorporated herein by reference in their entirety.

### FIELD

This disclosure relates generally to processes for enriching low-copy elements in DNA samples. More particularly, the present disclosure relates to the use of a methylation-dependent restriction endonuclease that recognizes methylated DNA and that cleaves upstream and downstream of the methylated DNA in processes for depleting repetitive methylated DNA elements and for enriching low-copy elements. The disclosed processes have particular utility in methods for analyzing features of low-copy elements with increased sensitivity.

### BACKGROUND

Genotypes can be readily described in terms of genetic markers. The most common form of genetic variation is a class of genetic variation known as a single nucleotide polymorphism (SNP). SNPs are important markers in many studies that link sequence variations to phenotypic changes. Hence, the identification of SNPs, also known as SNP-typing, is an important tool in molecular diagnostics and aims to determine at which positions at least one of the DNA bases differs from the reference sequence or from other genomes. Genotyping is the process of allele discrimination for an individual. The more genetic markers, the finer the definition of the genotype and the better it can be resolved from related genotypes.

SNP discovery can be done in several different ways, but whole genome sequencing and sequencing of complexity reduced genomic representations are the main approaches. To discover variation within and between populations, considering the high cost and complexity to sequence and assemble a complete genome, the whole genome sequencing method is currently only used in human studies, not in other species' populations.

Conventional genotyping is most often conducted using pre-defined SNP markers that must be discovered and validated in advance; these markers are often species or even population specific. These SNPs are typically detected via hybridization or by individual SNP-specific PCR-based assays. Genotyping by SNP array method is limited by the requirement for genomic variations to be clearly discovered before designing the array.

Several methods have been developed to reduce the sequencing effort and to restrict screening to a few thousand or several tens of thousands of single nucleotide polymorphisms (SNPs) at a highly reduced cost compared to whole genome sequencing or SNP-chip (array) analyses. These methods have been aiming at constructing reduced representation libraries (or RRLs) to reduce the complexity of the genome before sequencing, by enrichment for subsets of the genome either by capturing/targeting known fragments or by sequencing reproducible fraction of the genome sampled by application of restriction enzyme digestion. Examples of the later method include complexity reduction of polymorphic sequences (CRoPS), multiplexed shotgun genotyping, restriction-site associated DNA sequencing (RAD-seq) and genotyping-by-sequencing (e.g., DArTseq). All of these methods are based on a restriction enzyme digestion and adaptor ligation, followed by PCR and sequencing. The genotyping-by-sequencing (GBS) approach is widely used because it is straightforward, highly specific and reproducible; it typically achieves a few thousand to tens of thousands of SNPs depending on the heterogeneity of the population studied, the number of samples screened, and the applications envisaged. All of these genotyping methods suffer from a constraint in the number of markers they can generate. Only those fragments falling in a reasonable size range for PCR amplification and the sequencing technology being used can generate sequence data. Marker density can be an issue when many markers are needed to be employed in target regions.

A key contributor of genome complexity that undermines facile genomic analysis is the presence of repetitive DNA sequences which are a major component of eukaryotic genomes. Repetitive DNA sequences can be divided into minisatellite, microsatellite and satellite sequences. Satellite DNA sequences are considered to be a fast-evolving component of eukaryotic genomes, comprising tandemly-arrayed, highly-repetitive and highly-conserved monomer sequences. The monomer unit of satellite DNA is 150-400 base pairs (bp) in length. Repetitive sequences may be species- or genus-specific, and may be centromeric or subtelomeric in nature. They exhibit cohesive and concerted evolution caused by molecular drive, leading to high sequence homogeneity. Repetitive DNA sequences are present in all higher plants and can account for up to 90% of the genome size in some species. These repetitive DNA sequences are considered to generate major differences between genomes, which may reflect evolutionary distances between species. The repetitive sequences were once thought to be “selfish elements” or “junk DNA”, since they do not harbor genes.

### SUMMARY

The present disclosure is predicated in part on the determination that methylation-dependent restriction endonucleases that cleave upstream and downstream of a cognate methylated DNA recognition site can be used to deplete a sample of eukaryotic genomic DNA of repetitive DNA sequences (also referred to herein as “repetitive elements”) in which methylated DNA is over-represented relative to low copy sequences of the genomic DNA, to thereby enrich the abundance of low copy sequences (also referred to herein as “low copy elements”) in the sample. This depletion approach is useful for complexity reduction of genomic DNA analysis, including plant genomic DNA in which most DNA methylation is located in repetitive DNA sequences and low copy sequences are generally non-methylated or hypomethylated. The processes disclosed herein find particular utility in the analysis of genomic DNA, including analysis of genetic markers.

Accordingly, in one aspect of the present disclosure, processes are provided for reducing the level of repetitive elements in a DNA sample in which the repetitive elements comprise methylated DNA. These processes generally comprise, consist or consist essentially:
cleaving the DNA sample in the presence of a methylation-dependent restriction endonuclease that cleaves upstream and downstream of a cognate methylated DNA recognition site, to produce a population of cleavage products comprising a plurality of DNA fragments each comprising methylated DNA (also referred to herein as “methylated DNA fragments”); and
depleting the population of cleavage products of the plurality of methylated DNA fragments, thereby reducing the level of repetitive elements in the DNA sample.

In a related aspect of the present disclosure, processes are provided for enriching low-copy elements (e.g., non-repetitive elements) in a DNA sample. These processes generally comprise, consist or consist essentially:
cleaving the DNA sample in the presence of a methylation-dependent restriction endonuclease that cleaves upstream and downstream of a cognate methylated DNA recognition site, to produce a population of cleavage products comprising a plurality of methylated DNA fragments; and
depleting the population of cleavage products of the plurality of methylated DNA fragments, thereby enriching low copy elements in the DNA sample.

In specific embodiments, the methylation-dependent restriction endonuclease is an Mrr-like methylation-dependent restriction endonuclease (e.g., FspEI, MspJI, LpnPI, AspBHI, RIaI, SgrT, etc.). In representative embodiments, the methods further comprise performing a size selection process to separate the plurality of methylated DNA fragments from the DNA fragments that are not methylated (“non-methylated DNA fragments”) and that include low copy elements. Representative lengths of the methylated DNA fragments resulting from the cleavage may range from about 10 bp to about 50 bp, generally from about 20 bp to about 40 bp, and usually about 30 bp (e.g., 32 bp). In some embodiments, the low copy elements are about 50% or less (e.g., about 50% to about 1% and all integer percentages in between) as abundant as the repetitive DNA elements in the (starting) DNA sample before digestion.

The level or concentration of repetitive elements relative to low copy elements in the DNA sample following depletion of the methylated DNA fragments is decreased at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, as compared to the DNA sample before depletion of the methylated DNA fragments.

The level or concentration of low copy elements relative to repetitive elements in the DNA sample following depletion of the methylated DNA fragments is suitably increased by at least about 2-fold, at least about 3-fold, at least about 4-fold, at least about 5-fold, at least about 6-fold, at least about 7-fold, at least about 8-fold, at least about 9-fold, or at least about 10-fold, as compared to the DNA sample before depletion of the methylated DNA fragments.

The population of cleavage products may be depleted of the plurality of methylated DNA fragments by separating those fragments from the population using for example size selection or affinity selection, or by degrading the plurality of methylated DNA fragments using for example a methylation-dependent restriction enzyme that cleaves DNA at a cognate methylated recognition site (e.g., McrBC, DpnI and MspI). In illustrative examples of a size selection process, DNA fragments of about 50 bp or less, about 60 bp or less, about 70 bp or less, about 80 bp or less, about 90 bp or less, about 100 bp or less, about 110 bp or less, about 120 bp or less, about 130 bp or less, about 140 bp or less, about 150 bp or less, about 160 bp or less, about 170 bp or less, about 180 bp or less, about 190 bp or less, or about 200 bp or less comprising methylated DNA fragments are separated from the population of cleavage products in the size selection process. Suitably, the separation results in a low copy element-enriched fragment population in which DNA fragments are larger than about 200 bp.

In specific embodiments, the DNA sample is from a plant.

Yet another aspect of the present disclosure provides methods for analyzing DNA. These methods generally comprise, consist or consist essentially: providing a DNA sample that has a reduced level of repetitive elements and/or that is enriched in low copy elements, resulting from the repetitive element depletion/low copy element enrichment processes broadly described above and elsewhere herein, and an analyzing a feature of the DNA sample.

The feature may be a nucleotide sequence or genetic marker of the DNA sample. Nucleotide sequences may be analyzed, for example, by nucleic acid hybridization, nucleic acid amplification (e.g., polymerase chain reaction (PCR)), restriction digestion, and/or nucleotide sequencing (e.g., Sanger sequencing, pyrosequencing, nanopore sequencing and Next Generation sequencing). Representative examples of genetic markers include but are not restricted to single nucleotide polymorphisms (SNP), cleaved amplified polymorphic sequences (CAPS), deletion/insertion polymorphisms (DIP; also referred to as InDel mutations), copy number variants (CNV), short tandem repeats (STR), simple sequence repeats (SSR), random amplified polymorphic DNA (RAPD) markers, variable number of tandem repeats (VNTR), amplified fragment length polymorphisms (AFLP), retrotransposon-based insertion polymorphisms, sequence specific amplified polymorphism, quantitative trait loci (QTL), splicing variants, and haplotypes created from two or more of the aforementioned genetic markers.

### BRIEF DESCRIPTION OF THE DRAWINGS

FIG. 1 is photographic representation depicting size distribution of sorghum genomic DNA before and after MspJI digestion. Lane 1 is genomic DNA before digestion; Lane 2 is DNA after digestion, majority of DNA digested into fragments smaller than 100 bp.

FIG. 2 is photographic representation showing size distribution of sequencing library. Library ranged from 200 bp to 1200 bp.

FIG. 3 is photographic representation depicting size distribution of purified and size selected library. Fragments smaller than 300 bp are largely removed.

FIG. 4 is graphical representation showing a comparison of unique reads mapped to genic region between simulation of traditional GWS and DArTreseq in sorghum, wheat and oat.

FIG. 5 is graphical representation showing a comparison of sequencing depth of reads mapped to genic regions by using 10 million reads of both simulation and DArTreseq sample.

FIG. 6 is graphical representation depicting an integrative genomic viewer (IGV), which shows two sorghum sample reads (lane 1 and lane 2) aligning against sorghum 24 kb reference region on chromosome 1. There are 5 genes in this region which is indicated by five blue bars on the right (lane 3) of the figure. Each sample has 10 million DArTreseq reads. Majority of the reads are aligned in genic region, maximum coverage is 28 pair-end reads. This shows a very strong contrast to none-genic region which has very few reads.

FIG. 7 is graphical representation showing a heatmap (IGV) that illustrates one sorghum sample with 10 million DArTreseq reads aligned to reference genome. Left half of the picture is the over view of chromosome 1 and right half is the zoom in 388 kb of chromosome 1. Lane 1 in both heat maps is showing how many reads aligned to reference, the light grey color means no reads, the bluer of the color the more of the reads are aligned. Lane 2 in both heat maps shows the genic regions indicated by blue bars. This is another way to demonstrate DArTreseq technology enriched and sequenced single copy region, therefore, highly eliminated inter-genic high copy region and outstandingly avoided centromere.

### DETAILED DESCRIPTION

### I. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “an element” means one element or more than one element.

By “about” or “approximately” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The term “adaptor” refers to a nucleic acid that is ligatable to one or both strands of a double-stranded DNA molecule. In some embodiments, an adaptor may be a hairpin adaptor. In another embodiment, an adaptor may itself be composed of two distinct oligonucleotide molecules that are base paired with one another. As would be apparent, a ligatable end of an adaptor may be designed to be compatible with overhangs made by cleavage by a restriction enzyme, or it may have blunt ends.

The term “adaptor-ligated”, as used herein, refers to a nucleic acid that has been ligated to an adaptor. The adaptor can be ligated to a 5′ end and/or a 3′ end of a nucleic acid molecule.

As used herein, “and/or” refers to and encompasses any and all possible combinations of one or more of the associated listed items, as well as the lack of combinations when interpreted in the alternative (or).

The term “cleaving” or “cutting”, as used herein, refers to a reaction that breaks the phosphodiester bonds between two adjacent nucleotides in both strands of a double-stranded DNA molecule, thereby resulting in a double-stranded break in the DNA molecule. Accordingly, the term “cleavage site”, as used herein, refers to the site at which a double-stranded DNA molecule is or has been cleaved.

As used herein the term “cleavage product” refers to one or more new molecules produced as a result of cleavage or endonuclease activity by an enzyme.

The term “cognate” refers to two biomolecules that typically interact, including biomolecules that normally interact or co-exist in nature. Illustrative biomolecules of this type include an enzyme and its substrate, a restriction enzyme and its recognition site, and a receptor and its ligand.

“Complexity reduction” is used herein to denote a method wherein the complexity of a nucleic acid sample, such as genomic DNA, is reduced by the generation of a subset of the sample. This subset can be representative for the whole (i.e., complex) sample and is preferably a reproducible subset. By “reproducible” in this context is meant that when the same sample is reduced in complexity using the same method, the same, or at least comparable, subset is obtained. The method used for complexity reduction may be any method for complexity reduction known in the art. For example, if genetic material is isolated from same type of tissue at same growing stage, the complexity reduction methods as disclosed herein have in common that they are reproducible.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “DNA methylation” is used herein according to its meaning known in the art of molecular biology and molecular genetics; it refers to the addition of a methyl group to a specific base in the DNA. Typically, the methyl group is added to the 5 position of the cytosine (C) pyrimidine ring (typically abbreviated as “5mC”). DNA methylation is generally associated with gene repression and inactive chromatin. Methylation of DNA mostly takes place on cytosines, and in plants occurs both asymmetrically (mCpHpH) and symmetriccally (mCpG and mCpHpG).

The terms “DNA sample”, “sample DNA” and the like refer to a sample comprising DNA or nucleic acid representative of DNA isolated from a natural source. A DNA sample may comprise whole genomic sequences, part of a genomic sequence, chromosomal sequences, chloroplast sequences, and/or mitochondrial sequences, exons, long terminal repeat regions (LTR), intron regions, and regulatory sequences. These examples are not to be construed as limiting the sample types applicable to aspects of the present disclosure. A DNA sample may give rise to a population of nucleic acids in which a subset of the DNA molecules in the population may contain target sequences (e.g., low copy sequences) for enrichment. The population of DNA molecules may be for example: the product of random cleavage using enzymatic, mechanical or chemical means; the product of non-random or biased cleavage which is generally achieved with enzymes such as restriction enzymes; an appropriate size so that no cleavage or fragmentation is required; or a product of environmental damage. In specific embodiments, the DNA sample is from a plant or plant part.

As used herein, the term “depletion” and its grammatical equivalents refer to the result of any process which decreases the level or concentration in a DNA sample of non-target nucleic acid (e.g., repetitive element) relative to target nucleic acid (e.g., low copy element, allele, genetic marker, etc.), as compared to a corresponding DNA sample not subjected to the process. In specific embodiments, the level or concentration of non-target nucleic acid relative to target nucleic acid in a DNA sample subjected to a depletion process is decreased at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, as compared to a corresponding DNA sample not subjected to the depletion process.

As used herein, the term “enrichment” and its grammatical equivalents refer to the result of any process which increases the level or concentration in a DNA sample of target nucleic acid (e.g., low copy element, genetic marker, etc.) relative to non-target nucleic acid (e.g., repetitive element), as compared to a corresponding DNA sample not subjected to the process. In specific embodiments, the level or concentration of target nucleic acid relative to non-target nucleic acid in a DNA sample subjected to an enrichment process is increased by at least about 2-fold, at least about 3-fold, at least about 4-fold, at least about 5-fold, at least about 6-fold, at least about 7-fold, at least about 8-fold, at least about 9-fold, or at least about 10-fold, as compared to a corresponding DNA sample not subjected to the enrichment process.

As used herein, the term “genic region” refers to a nucleic acid sequence that codes for at least one RNA and/or polypeptide. The genic region may also encompass any identifiable adjacent 5′ and 3′ non-coding nucleotide sequences involved in the regulation of expression of a protein or a non-protein-coding RNA up to about 2 kb upstream of the coding region and 1 kb downstream of the coding region, but possibly further upstream or downstream. A genic region further includes any introns that may be present in the genic region. Further, the genic region may comprise a single gene sequence, or multiple gene sequences interspersed with short spans (less than 1 kb) of non-genic sequences.

As used herein, a “genic” sequence is a nucleic acid sequence that encodes a protein or a non-protein-coding RNA. A genic sequence can include one or more introns. By contrast, a “non-genic” sequence as used herein is a nucleic acid sequence that is not a genic sequence

“Genotyping”, as used herein, refers to the process of determining genetic variations among individuals in a species. The genotype of an organism is the inherited instructions it carries within its genetic code. SNPs are the most common type of genetic variation and by definition are single-base differences at a specific locus that is found in more than 1% of the population. SNPs are found in both coding and non-coding regions of the genome and can lead to different phenotypes, such as the ability to get a disease or to have resistance against it, when found in coding regions. Hence, SNPs are often used as markers for certain diseases or some phenotypes. When found in non-coding regions, SNPs act as markers for evolutionary genomics studies. Related to SNPs are “InDels” or insertions and deletions of nucleotides of varying length. A third type of genetic variation is copy number variation (CNV), which results from having different numbers of copies of a DNA segment in various genomes.

The term “hypermethylation” refers to the average methylation state corresponding to an increased presence of methylated nucleotides. In some embodiments, the hypermethylation corresponds to an increase of 5mC at one or a plurality of CpG dinucleotides.

The term “hypomethylation” refers to the average methylation state corresponding to an decreased presence of methylated nucleotides. In some embodiments, the hypermethylation corresponds to a decrease of 5mC at one or a plurality of CpG dinucleotides.

As used herein a “locus” is a position on a chromosome where a gene or marker or allele is located. In some embodiments, a locus may encompass one or more nucleotides.

The term “low-copy” or “low-copy number” element or nucleic acid as used herein refers to a species of nucleic acid, for example a genic region, genic sequence or genetic marker, that is present in relatively lower proportion than other species of nucleic acid (e.g., repetitive elements) in a population of nucleic acids. That is, the abundance of a low-copy element or nucleic acid is lower in proportion than the abundance of a non-low-copy element nucleic acid in a population of nucleic acids. In one example, a low-copy element or nucleic acid refers to the fraction or proportion of a genic region in a population of nucleic acids containing non-genic regions such as repetitive DNA sequences. The person of ordinary skill will further appreciate that enrichment of a low-copy nucleic acid as referred to herein indicates increasing the proportion or the fraction of the low-copy nucleic acid relative to the population of nucleic acids.

As used herein, the terms “marker”, “molecular marker” and “genetic marker” are used interchangeably to refer to a nucleotide and/or a nucleotide sequence that has been associated with a phenotype, trait or trait form. In some embodiments, a marker may be associated with an allele or alleles of interest and may be indicative of the presence or absence of the allele or alleles of interest in a cell or organism. A marker may be, but is not limited to, an allele, a gene, a haplotype, a restriction fragment length polymorphism (RFLP), a simple sequence repeat (SSR), random amplified polymorphic DNA (RAPD), cleaved amplified polymorphic sequences (CAPS), an amplified fragment length polymorphism (AFLP), a single nucleotide polymorphism (SNP), a sequence-characterized amplified region (SCAR), a sequence-tagged site (STS), a single-stranded conformation polymorphism (SSCP), an inter-simple sequence repeat (ISSR), an inter-retrotransposon amplified polymorphism (IRAP), a retrotransposon-microsatellite amplified polymorphism (REMAP), a chromosome interval, or an RNA cleavage product (such as a Lynx tag). A marker may be present in genomic or expressed nucleic acids (e.g., ESTs). The term marker may also refer to nucleic acids used as probes or primers (e.g., primer pairs) for use in amplifying, hybridizing to and/or detecting nucleic acid molecules according to methods well known in the art. Markers corresponding to genetic polymorphisms between members of a population can be detected by methods well-established in the art. These include, e.g., nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), and/or detection of amplified fragment length polymorphisms (AFLPs). Well established methods are also known for the detection of expressed sequence tags (ESTs) and SSR markers derived from EST sequences and randomly amplified polymorphic DNA (RAPD).

As used herein, the term “marker assisted selection (MAS)” refers to a process whereby organism such as plants are screened for the presence and/or absence of one or more genetic and/or phenotypic markers in order to accelerate the transfer of the DNA region comprising the marker (and optionally lacking flanking regions) into an (elite) breeding line. In other words, “MAS” is a process of using the presence of molecular markers, which are genetically linked to a particular locus or to a particular chromosome region (e.g., introgression fragment), to select organism such as plants for the presence of the specific locus or region (introgression fragment).

The term “methylation state” refers to the presence or absence of 5mC at one or a plurality of CpG dinucleotides within a DNA sequence. Methylation states at one or more particular CpG methylation sites (each having two antiparallel CpG dinucleotide sequences) within a DNA sequence include “unmethylated,” “fully-methylated” and “hemi-methylated.”

As used herein the term “Mrr-like methylation-dependent restriction endonuclease” refers to a restriction enzyme that is dependent on methylation or hydroxymethylation for cleavage to occur. Representative enzymes of this type include, but are not restricted to, MspJI, FspEI, LpnPI, AspBHI, RIaI and SgrT. For example, MspJI recognizes 5mC in the context of its recognition site mCNNR (wherein R=G or A) and introduces double-stranded breaks at fixed distances (N12/N16 from mC) on the 3′ side of the mC, leaving a four-base 5′ overhang. A unique feature of these enzymes is that with symmetrically methylated sequences [e.g., mCpG or mCHG sites, (wherein H=C, T, or A)], cleavages elicited by two methylated sites on opposite strands result in DNA fragments of approximately 32 bp long being excised from the genomic DNA, with the methylated recognition site located at about the middle of the excised fragments.

The terms “nucleic acid”, “nucleotide sequence” “nucleic acid sequence” “nucleic acid molecule”, “oligonucleotide” and “polynucleotide” are used interchangeably herein to refer to a heteropolymer of nucleotides and encompass both RNA and DNA, including cDNA, genomic DNA, mRNA, synthetic (e.g., chemically synthesized) DNA or RNA and chimeras of RNA and DNA. Nucleic acid can be single-stranded or double-stranded. Where single-stranded, the nucleic acid can be a sense strand or an antisense strand. In specific embodiments, the nucleic acid is double-stranded DNA. As used herein, the term “DNA fragment” refers to a fraction of a given DNA molecule.

As used herein, the terms “phenotype”, “phenotypic trait” or “trait” refer to one or more traits and/or manifestations of an organism such as its morphology, development, biochemical or physiological properties, phenology, behavior, and products of behavior. The phenotype can be a manifestation that is observable to the naked eye, or by any other means of evaluation known in the art, e.g., microscopy, biochemical analysis, or an electromechanical assay. Phenotypes may result from the expression of the genes of an organism as well as the influence of environmental factors and the interactions between the two. In some cases, a phenotype or trait is directly controlled by a single gene or genetic locus, i.e., a “single gene trait”. In other cases, a phenotype or trait is the result of several genes.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant element” is synonymous to a “portion” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture. Representative plants contemplated in the present disclosure include acacia, alfalfa, algae, aneth, apple, apricot, artichoke, arugula, asparagus, avocado, banana, barley, beans, beech, beet, Bermuda grass, bent grass, blackberry, blueberry, Blue grass, broccoli, Brussels sprouts, cabbage, camelina, canola, cantaloupe, carinata, carrot, cassava, cauliflower, celery, cherry, chicory, cilantro, citrus, clementines, coffee, corn, cotton, cucumber, duckweed, Douglas fir, eggplant, endive, escarole, eucalyptus, fennel, fescue, figs, forest trees, garlic, gourd, grape, grapefruit, honey dew, jicama, kiwifruit, lettuce, leeks, lemon, lime, Loblolly pine, maize, mango, melon, mushroom, nectarine, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, seaweed, scallion, sorghum, Southern pine, soybean, spinach, squash, strawberry, sudangrass, sugar beet, sugarcane, sunflower, sweet potato, sweetgum, switchgrass, tangerine, tea, tobacco, tomato, triticale, turf, turnip, a vine, watermelon, wheat, yams, and zucchini.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

As used herein a “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

As used herein, the term “polymorphism” refers to a variation in the nucleotide sequence at a locus, where said variation is too common to be due merely to a spontaneous mutation. A polymorphism can be a SNP or an insertion/deletion polymorphism, also referred to herein as an “indel”. The polymorphic site or sites of a nucleotide sequence can be determined by comparing the nucleotide sequences at one or more loci in two or more DNA samples from different organisms or from the same organism.

As used herein, “quantitative trait locus (QTL)” refers to a locus that controls to some degree numerically representable traits that are usually continuously distributed.

The term “repetitive elements”, “repetitive sequences”, “repetitive genomic sequences” and the like are used interchangeably herein to generally refer to long sequence stretches that occur two or more times in the genome with high similarity between occurrences. For example, a repetitive sequence may appear multiple times in a region of the DNA, separated by the different DNA sequences. For example, repetitive sequences may be categorized in sequence families and may be broadly classified as interspersed repetitive or tandemly repeated DNA. Interspersed repetitive sequences comprise copies of transposable elements interspersed throughout the genome, some of which are still active and are often referred to as “jumping genes”. There are at least two classes of interspersed repetitive elements: Class I elements and Class II elements. Class I elements (or “retroelements”—such as retrotransposons, long interspersed nucleotide elements and short interspersed nucleotide elements, for example) transpose via reverse transcription of an RNA intermediate. Class II elements (or DNA transposable elements—such as transposons, Tn elements, insertion sequence elements and mobile gene cassettes of bacterial integrons, for example) transpose directly from one site in the DNA to another. Tandem repeat sequences refer to copies of DNA sequences that lie adjacent to each other in the same orientation (direct tandem repeats) or in the opposite direction to each other (inverted tandem repeats). Repetitive sequences may include satellite, minisatellite, and microsatellite DNA. Generally, microsatellites comprise 2-5 bp repeats and an array size of the order of 10-100 units, minisatellites comprise 6-100 bp (usually around 15 bp) repeats and an array size of 0.5-30 kb, and satellite DNA (satDNA) comprise a variable AT-rich repeat unit that often forms arrays up to 100 Mb. In some cases, a repetitive sequence may be a segment of DNA that contains a sequence of nucleotides that is repeated for at least 3, 5, 10, 15, 20, 30, 40, 50, 60, 80, or 100 or more times. The major types of repetitive elements in plant genomes include transposable elements (TEs), simple sequence repeats (SSRs), and ribosomal DNA.

The term “restriction endonuclease” or “restriction enzyme” is intended to refer to an enzyme that recognizes a specific recognition site (or restriction site) on a single-stranded or double-stranded nucleic acid molecule and cuts this molecule at a cleavage site. As used herein, the term “recognition site” refers to a specific sequence of nucleotides recognized by a restriction enzyme. As used herein, the term “cleavage site” refers to the site wherein the restriction enzyme cleaves the nucleic acid molecule. Restriction enzymes may recognize and cleave nucleic acid molecule at the same site. Restriction enzymes may also cleave nucleic acid molecule at a site distant from the recognition site. According to the restriction enzyme, the cleavage site may be located downstream and/or upstream to the recognition site.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide are obtained. The term “next-generation sequencing” refers to the so-called parallelized sequencing-by-synthesis or sequencing-by-ligation platforms currently employed by Illumina, Life Technologies, and Roche etc. Next-generation sequencing methods may also include nanopore sequencing methods or electronic-detection based methods such as Ion Torrent technology commercialized by Life Technologies.

Each embodiment described herein is to be applied mutatis mutandis to each and every embodiment unless specifically stated otherwise.

### 2. Abbreviations

The following abbreviations are used throughout the application:

| Abbreviation | Definition |
|---|---|
| bp | Base pairs |
| GBS | Genotyping-by-sequencing |
| GWS | Genome-wide selection |
| kb | Kilobase(s) or kilobase pair(s) |
| NGS | Next-generation sequencing |
| SNP | Single nucleotide polymorphism |
| SSR | Simple sequence repeat |
| WGS | Whole genome sequencing |

### 3. Methods for Depleting Repetitive Elements for Enrichment of Low-Copy Elements in a DNA Sample

Disclosed herein are methods for enriching selected nucleic acids to enhance downstream analytical methods. Central to the discriminatory enrichment of a subset of nucleic acids (“target nucleic acids”) that are often low-copy number nucleic acids is the utilization of a methylation-dependent restriction endonuclease that cleaves upstream and downstream of a cognate methylated nucleic acid recognition site in “non-target” methylated repetitive sequences, to produce a population of cleavage products comprising a plurality of methylated nucleic acid fragments. These methylated nucleic acid fragments are separated from the cleavage product to thereby produce a nucleic acid sample that is depleted in non-target repetitive sequences and enriched in target low-copy nucleic acids. Enrichment of such target nucleic acids enhances downstream applications including analysis of nucleotide sequence and genetic markers in low-copy nucleic acids.

One embodiment of the present disclosure provides processes for reducing the level of repetitive elements in a DNA sample in which the repetitive elements comprise methylated DNA, suitably for enriching low-copy elements (e.g., non-repetitive elements) in the DNA sample, wherein the methods comprise, consists or consist essentially of: cleaving the DNA sample in the presence of a methylation-dependent restriction endonuclease that cleaves upstream and downstream of a cognate methylated DNA recognition site, to produce a population of cleavage products comprising a plurality of methylated DNA fragments; and depleting the population of cleavage products of the plurality of methylated DNA fragments, thereby reducing the level of repetitive elements and suitably enriching low copy elements in the DNA sample.

The depletion/enrichment processes are typically performed in vitro, i.e., in a cell-free environment using isolated genomic DNA. The genomic DNA may be isolated from any source, including any organism, organic material or nucleic acid-containing substance can be used as a source of nucleic acids to be processed in accordance with the disclosed processes. In certain embodiments, the genomic DNA may be derived from a plant, e.g., rice, sorghum, cowpea, wheat, oat, barley and maize. Methods of preparing genomic DNA for analysis is routine and known in the art, such as those described by Ausubel, F. M. et al., (Short protocols in molecular biology, 3rd ed., 1995, John Wiley & Sons, Inc., New York) and Sambrook, J. et al. (Molecular cloning: A laboratory manual, 2.sup.nd ed., 1989, Cold Spring Harbor Laboratory Press, Cold Spring Harbor, N.Y.). In certain cases, the sample used may contain total genomic DNA, or genomic DNA found in the nucleus (nuclear genomic DNA), or a subcellular component DNA (e.g., mitochondrial genomic DNA, plastid genomic DNA, chloroplast genomic DNA, apicoplast genomic DNA, or mixtures thereof). The genomic DNA may or may not be already fragmented by other means, e.g., fragmented into fragments that are over 10 kb, or over 50 kb in length.

Any suitably methylation-dependent restriction endonuclease that cleaves upstream and downstream of a cognate methylated DNA recognition site can be used in the disclosed processes. In preferred embodiments, the methylation-dependent restriction endonuclease is an Mrr-like methylation-dependent restriction endonuclease (e.g., FspEI, MspJI, LpnPI, AspBHI, RIaI, SgrT, etc.).

The population of cleavage products may be depleted of methylated DNA fragments by ay suitable procedure. For example, the methylated DNA fragments may be separated from the population of cleavage products by size selection process using for example gel electrophoresis, gel purification, liquid chromatography, size exclusion purification, filtration purification methods, and/or bead-based separation techniques, among others. In some embodiments, DNA fragments of about 50 bp or less, about 60 bp or less, about 70 bp or less, about 80 bp or less, about 90 bp or less, about 100 bp or less, about 110 bp or less, about 120 bp or less, about 130 bp or less, about 140 bp or less, about 150 bp or less, about 160 bp or less, about 170 bp or less, about 180 bp or less, about 190 bp or less, or about 200 bp or less comprising the methylated DNA fragments corresponding to the repetitive elements are separated from the population of cleavage products in the size selection process. In preferred embodiments, the separation results in a low copy element-enriched fragment population in which DNA fragments are larger than about 200 bp.

In other embodiments, the population of cleavage products is depleted of methylated DNA fragments by methylation affinity separation using for example a reagent that has affinity for methylated CpG dinucleotides, representative examples of which include methyl-CpG binding domain (MBD) proteins (e.g., MethylMagnet GST-MBD2 fusion protein).

In still other embodiments, depletion of methylated DNA fragments may be achieved by degrading the methylated DNA with a methylation-dependent restriction enzyme that cleaves DNA at a cognate methylated recognition site, non-limiting examples of which include, McrBC, DpnI, HpaII and MspI.

In some cases, the population of cleavage products that is enriched in fragments comprising low copy elements may be cloned into a vector, e.g., a fosmid, bac or cosmid vector for storage and later analysis. This low copy element-enriched fragment population may be subjected to one or more fragmentation reactions for cloning and/or analysis of the low copy elements. The low copy element-enriched fragment population may be fragmented by sonication, needle shear, nebulization, shearing (e.g., acoustic shearing, mechanical shearing, point-sink shearing), passage through a French pressure cell, enzymatic digestion and transposon-mediated fragmentations. Fragmentation of the low copy element-enriched fragment population may result in fragment sized of about 100 base pairs to about 2000 base pairs, about 200 base pairs to about 1500 base pairs, about 200 base pairs to about 1000 base pairs, about 200 base pairs to about 500 base pairs, about 500 base pairs to about 1500 base pairs, and about 500 base pairs to about 1000 base pairs. The one or more fragmentation reactions may result in fragment sized of about 50 base pairs to about 1000 base pairs. The one or more fragmentation reactions may result in fragment sized of about 100 base pairs, 150 base pairs, 200 base pairs, 250 base pairs, 300 base pairs, 350 base pairs, 400 base pairs, 450 base pairs, 500 base pairs, 550 base pairs, 600 base pairs, 650 base pairs, 700 base pairs, 750 base pairs, 800 base pairs, 850 base pairs, 900 base pairs, 950 base pairs, 1000 base pairs or more.

In some cases, the fragments may be treated with Taq polymerase to produce 3′ A overhangs, and then cloned by TA cloning. In some cases, the fragments may be amplified prior to cloning and/or analysis, which may involve ligating adaptors onto the ends of the fragments, and amplifying the fragments using primers that hybridize to the ligated adaptors. The fragments (whether or not they are cloned in a vector) may be analyzed by a suitable DNA analysis method which analyze a feature of the DNA. The feature may be a nucleotide sequence or genetic marker of the DNA.

In particular embodiments, the fragments are sequenced. For example, the fragments may be sequenced by next generation sequencing (NGS) technologies, representative examples of which include the 454 Life Sciences platform (Roche, Branford, Conn.) (Margulies et al. 2005 Nature, 437, 376-380); IIlumina's Genome Analyzer, GoldenGate Methylation Assay, or Infinium Methylation Assays, i.e., Infinium HumanMethylation 27K BeadArray or VeraCode GoldenGate methylation array (Illumina, San Diego, Calif.; Bibkova et al. 2006, Genome Res. 16, 383-393; U.S. Pat. Nos. 6,306,597 and 7,598,035 (Macevicz); U.S. Pat. No. 7,232,656 (Balasubramanian et al.)); QX200™ Droplet Digital™ PCR System from Bio-Rad; or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453 (Barany et al.); the Helicos True Single Molecule DNA sequencing technology (Harris et al., 2008 Science, 320, 106-109; U.S. Pat. Nos. 7,037,687 and 7,645,596 (Williams et al.); U.S. Pat. No. 7,169,560 (Lapidus et al.); U.S. Pat. No. 7,769,400 (Harris)), the single molecule, real-time (SMRT™) technology of Pacific Biosciences, and sequencing (Soni and Meller, 2007, Clin. Chem. 53: 1996-2001); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys). These systems allow the sequencing of many nucleic acid molecules isolated from a specimen at high orders of multiplexing in a parallel fashion. Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

In certain embodiments, the fragment may be amplified using primers that are compatible with use in, e.g., Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al. (Nature 2005 437: 376-80); Ronaghi et al. (Analytical Biochemistry 1996 242: 84-9); Shendure et al. (Science 2005 309: 1728-32); Imelfort et al. (Brief Bioinform. 2009 10:609-18); Fox et al. (Methods Mol Biol. 2009; 553:79-108); Appleby et al. (Methods Mol Biol. 2009; 513:19-39) and Morozova et al. (Genomics 2008 92:255-64), which are incorporated by reference for the general descriptions of the methods and the particular steps of the methods, including all starting products, reagents, and final products for each of the steps. In some cases, the fragments may be subjected to target enrichment methods prior to sequencing. Target enrichment methods are known in the art and encompass methods such as SureSelect and HaloPlex technologies commercialized by Agilent Technologies, PCR-amplification based strategies, and the like.

In some embodiments, the fragments are sequenced using nanopore sequencing (e.g. as described in Soni et al., 2007 Clin Chem 53: 1996-2001, or as described by Oxford Nanopore Technologies). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology is disclosed in U.S. Pat. Nos. 5,795,782, 6,015,714, 6,627,067, 7,238,485 and 7,258,838 and U.S. Pat Appln Nos. 2006003171 and 20090029477.

In some embodiments, the fragments can be analyzed for genetic markers, non-limiting examples of which include single nucleotide polymorphisms (SNP), cleaved amplified polymorphic sequences (CAPS), deletion/insertion polymorphisms (DIP; also referred to as InDel mutations), copy number variants (CNV), short tandem repeats (STR), simple sequence repeats (SSR), random amplified polymorphic DNA (RAPD) markers, variable number of tandem repeats (VNTR), amplified fragment length polymorphisms (AFLP), retrotransposon-based insertion polymorphisms, sequence specific amplified polymorphism, quantitative trait loci (QTL), splicing variants, and haplotypes created from two or more of the aforementioned genetic markers.

As will be appreciated, the present disclosure facilitates many different genetic studies, such as breading trait selection, genomic mapping, establishing relatedness of germplasm, etc., and thus breeding programs will benefit particularly but not exclusively from this disclosure. For example, high SNP density in gene region and other single copy regions makes so many available markers for breeders to identify the functional variation that is responsible for traits. Ecologists will also benefit from the present disclosure through obtaining high density genome profiles to establish their population structure and their evolutionary patterns.

In order that the disclosure may be readily understood and put into practical effect, particular preferred embodiments will now be described by way of the following non-limiting examples.

### EXAMPLES

### Example 1

### Enrich Low Copy Number Genomic Regions

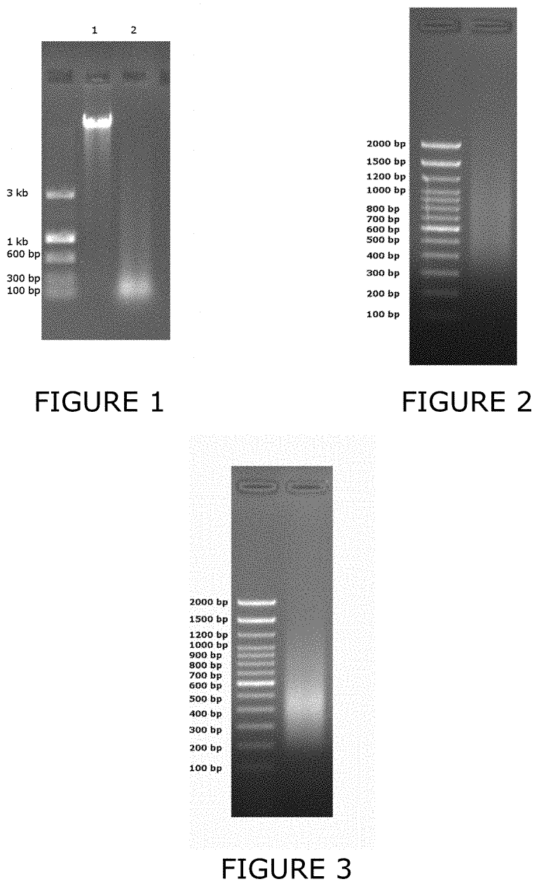

Representative samples of sorghum, wheat and oat germplasms are identified for genotyping. The samples are chosen solely for demonstration purposes. DNA was extracted by using a NucleoMag 96 Tissue Kit (MachereyNagel, Duren, Germany) coupled with NucleoMag SEP (Ref. 744900) to allow automated separation of high-quality DNA on a Freedom Evo robotic liquid handler (TECAN, Miinnedorf, Switzerland). Sorghum, wheat and oat genomic DNA was extracted from young seedlings. Tissue was snap freeze in liquid nitrogen and crashed by using SPEX SamplePrep Geno/Grinder (SPEXSamplePrep, Metuchen, USA) with the help of a stainless-steel ball inside the sample tube. About 1 μg of DNA from each sample is digested at 37° C. for 4 hours with 10 units of MspJI (New England Biolabs, Massachusetts, USA), 5 μL of 10×NEB buffer, 1 μL enzyme activator solution in a volume of 50 μL. After that, stop the digestion by increase temperature to 65° C. for 20 min. To check the digestion, load 5 μL of DNA on a 1.5% TAE agarose gel (FIG. 1). DNA is purified with 0.8 volume of AMPure beads (Beckman Coulter, California, USA), and eluted in 15 μL of EB buffer (QIAGEN, Hilden, Germany). DNA fragments larger than 300 bp are retained and purified (FIG. 2).

### Example 2

### Constructing Library Using Enriched Unmethylated Genomic DNA

All the purified DNA is used to make library by using commercially available Nextera DNA Flex Library Prep kit (Illumina, California, USA), following the kit manual on Illumina website. Each library has unique dual-barcode. After PCR, check 3 μL of PCR products on a 2% TAE agarose gel. About 120 samples are equally pooled, and pooled DNA is purified with 0.7 volume of Ampure beads. After purification, load 3 μL on a 2% TAE gel (FIG. 3) to check the size distribution.

### Example 3

### Sequencing Complexity Reduced Library and Analyzing Data

Sequencing was performed on a 150 cycles pair-end run on MGI2000 sequencer (BGI, Shenzhen, China) to achieve 10 to 20 million reads per sample. Raw sequence reads were processed in DArTreseq pipeline. Poor quality reads were discarded. Sequences confirming to our standards align to reference genome. Sorghum_v301, wheat_ChineseSprong10 and Oat_OT3098_v1 are used as references for 3 different genome sequence alignment. Overall alignment rate is 85% of the filtered reads.

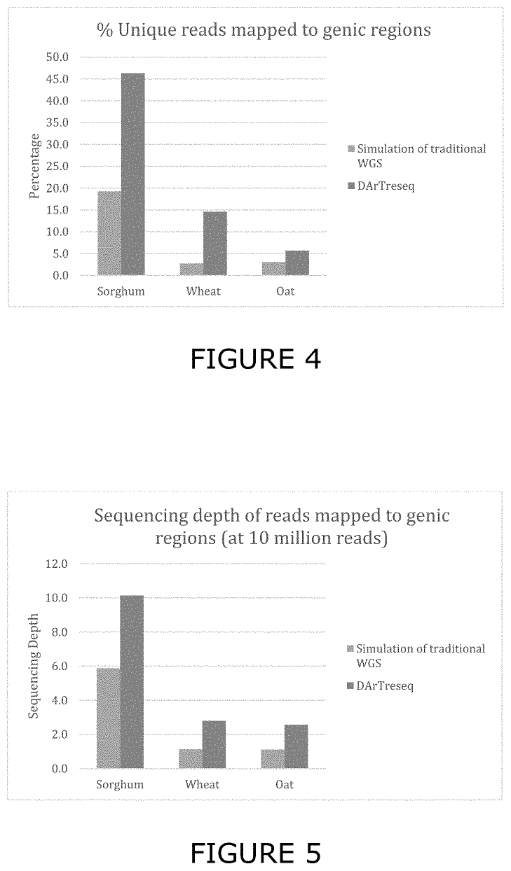

The DArTreseq technology is able to concentrate reads from genomic regions with low DNA methylation levels, which usually are genic regions in plant genomes. Reads from genic regions are considered as high-quality reads for multiple down streaming analyses, especially for genotyping. In a small genome crop, sorghum, more than 45% of total sequencing reads are uniquely mapped to genic regions using DArTreseq. As a comparison, this number is predicted to be only 20% if using traditional WGS method. This means that higher percentage of sequencing data can actually be used. DArTreseq performs even better in large genome crops like wheat. There is an over 4 times increase in DArTreseq samples compared to traditional WGS samples (FIG. 4).

Due to concentrated sequencing reads in genic regions, DArTreseq is able to achieve high read depths with low sequencing throughputs compared to traditional WGS. Data showed average read depth in genic regions is increased by 0.7 time in sorghum and 1.5 times in wheat (FIG. 5). This significantly reduces the costs of genotyping especially for large genome species.

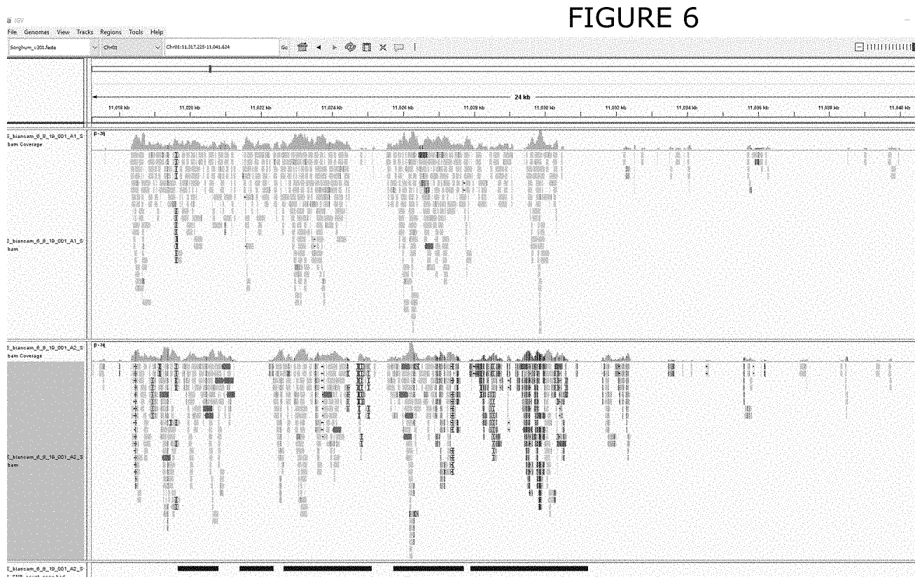

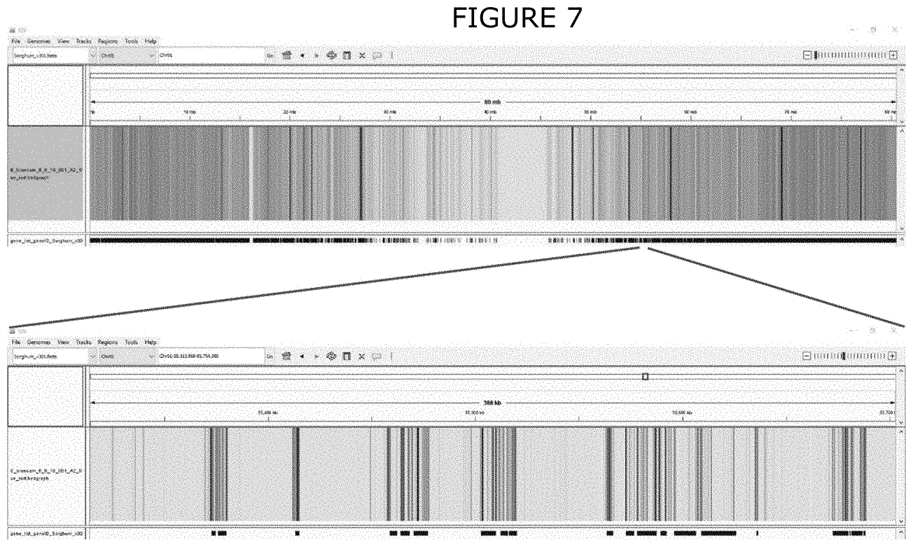

Results show a reduction of the genomic complexity in none gene regions. SNP identification was visualized in the integrative genomic viewer (IGV) (FIG. 6). The efficiency of the enrichment is shown as majority of reads are aligned in or close to gene, none gene regions are either not covered or covered by very few reads. Heatmap (IGV) provides an over view of reads align to genic and none-genic region (FIG. 7).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

What is claimed is:

1. A process for reducing the level of repetitive elements in a DNA sample in which the repetitive elements comprise methylated DNA or for enriching low-copy elements, the process comprising:
cleaving the DNA sample in the presence of a methylation-dependent restriction endonuclease that cleaves upstream and downstream of a cognate methylated DNA recognition site, to produce a population of cleavage products comprising a plurality of DNA fragments each comprising methylated DNA (“methylated DNA fragments”); and
depleting the population of cleavage products of the plurality of methylated DNA fragments, thereby reducing the level of repetitive elements or enriching low copy elements in the DNA sample.

2. The process of claim 1, wherein the methylation-dependent restriction endonuclease is an Mrr-like methylation-dependent restriction endonuclease.

3. The process of claim 2, wherein the Mrr-like methylation-dependent restriction endonuclease is selected from FspEI, MspJI, LpnPI, AspBHI, RIaI and SgrT.

4. The process of claim 2, wherein the Mrr-like methylation-dependent restriction endonuclease is MspJI.

5. The process of claim 1, wherein the population of cleavage products is depleted of the plurality of methylated DNA fragments by separating the plurality of methylated DNA fragments from the population of cleavage products.

6. The process of claim 5, wherein the plurality of methylated DNA fragments is separated from the population of cleavage products by a size selection process.

7. The process of claim 6, wherein the size selection process is selected from gel electrophoresis, gel purification, liquid chromatography, size exclusion purification, filtration purification methods and bead-based separation techniques.

8. The process of claim 1, wherein DNA fragments of about 200 bp or less comprising methylated DNA fragments are separated from the population of cleavage products in the size selection process.

9. The process of claim 1, wherein the separation results in a low copy element-enriched fragment population in which DNA fragments are larger than about 200 bp.

10. The process of claim 5, wherein the plurality of methylated DNA fragments is separated from the population of cleavage products by methylation affinity separation.

11. The process of claim 10, wherein the methylation affinity separation is carried out using a reagent that has affinity for methylated CpG dinucleotides.

12. The process of claim 5, wherein the plurality of methylated DNA fragments is separated from the population of cleavage products by degrading the methylated DNA with a methylation-dependent restriction enzyme that cleaves DNA at a cognate methylated recognition site.

13. The process of claim 12, wherein the methylation-dependent restriction enzyme is selected from McrBC, DpnI, HpaII and MspI.

14. The process of claim 1, wherein the level or concentration of repetitive elements relative to low copy elements in the DNA sample following depletion of the methylated DNA fragments is decreased at least about 5%, as compared to the DNA sample before depletion of the methylated DNA fragments, or wherein the level or concentration of low copy elements relative to repetitive elements in the DNA sample following depletion of the methylated DNA fragments is increased by at least about 2-fold, as compared to the DNA sample before depletion of the methylated DNA fragments.

15. The process of claim 1, wherein the DNA sample is from a plant.

16. The process of claim 15, wherein the plant is selected from acacia, alfalfa, algae, aneth, apple, apricot, artichoke, arugula, asparagus, avocado, banana, barley, beans, beech, beet, Bermuda grass, bent grass, blackberry, blueberry, Blue grass, broccoli, Brussels sprouts, cabbage, camelina, canola, cantaloupe, carinata, carrot, cassava, cauliflower, celery, cherry, chicory, cilantro, citrus, clementines, coffee, corn, cotton, cucumber, duckweed, Douglas fir, eggplant, endive, escarole, eucalyptus, fennel, fescue, figs, forest trees, garlic, gourd, grape, grapefruit, honey dew, jicama, kiwifruit, lettuce, leeks, lemon, lime, Loblolly pine, maize, mango, melon, mushroom, nectarine, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, rye grass, seaweed, scallion, sorghum, Southern pine, soybean, spinach, squash, strawberry, sudangrass, sugar beet, sugarcane, sunflower, sweet potato, sweetgum, switchgrass, tangerine, tea, tobacco, tomato, triticale, turf, turnip, a vine, watermelon, wheat, yams, and zucchini.

17. A method for analyzing DNA, the method comprising providing a DNA sample that has a reduced level of repetitive elements that comprise methylated DNA and/or that is enriched in low copy elements, wherein the DNA sample is produced by the process of claim 1, and analyzing a feature of the DNA sample.

18. The method of claim 17, wherein the feature is a nucleotide sequence of the DNA sample.

19. The method of claim 21, wherein the nucleotide sequence is analyzed by nucleic acid hybridization, nucleic acid amplification, restriction digestion and/or nucleotide sequencing.

20. The method of claim 17, wherein the feature is a genetic marker of the DNA sample.

21. The method of claim 20, wherein the genetic marker is selected from single nucleotide polymorphisms (SNP), cleaved amplified polymorphic sequences (CAPS), deletion/insertion polymorphisms (DIP; also referred to as InDel mutations), copy number variants (CNV), short tandem repeats (STR), simple sequence repeats (SSR), random amplified polymorphic DNA (RAPD) markers, variable number of tandem repeats (VNTR), amplified fragment length polymorphisms (AFLP), retrotransposon-based insertion polymorphisms, sequence specific amplified polymorphism, quantitative trait loci (QTL), splicing variants, and haplotypes created from two or more of the aforementioned genetic markers.
